(19) United States
(12) Patent Application Publication
(10) Pub. No.: US 2025/0288272 A1
(43) Pub. Date: Sep. 18, 2025

(54) X-RAY MACHINES WITH INTEGRATED SENSORS FOR REDUCING OR PREVENTING RADIATION INJURY

(71) Applicant: Ziehm Imaging GmbH, Nuremberg (DE)

(72) Inventors: Markus Mronz, Eckental (DE); Ewald Hauser, Leinburg (DE); Thomas Sander, Nuremberg (DE)

(21) Appl. No.: 19/074,116

(22) Filed: Mar. 7, 2025

(30) Foreign Application Priority Data
Mar. 15, 2024 (DE) 102024000861.6

(51) Int. Cl. A61B 6/00; A61B 6/04

(52) U.S. Cl. CPC A61B 6/542; A61B 6/0407; A61B 6/547

(57) ABSTRACT

A medical imaging system, such as an x-ray machine or a C-arm, is configured to enhance the safety of patients during an imaging procedure. The system can include a C-arm X-ray machine for generating X-ray radiation, a table or patient support surface configured to position the patient during the imaging procedure, a distance sensor configured to measure the distance between the C-arm X-ray device and/or generator and/or C-arm housing and/or the patient and/or the table, a dose control unit configured to control the radiation dose of the X-ray beam based on the measured distances between the C-arm X-ray device and the patient, and a skin protection system configured to interrupt and/or minimize the radiation dose in critical situations while maintaining sufficient image quality by adjusting the radiation intensity in real time to limit radiation exposure of the patient's skin.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### BACKGROUND

### Field

The present disclosure generally relates to the field of medical imaging devices and methods, and more specifically to X-ray machines with integrated sensors to reduce or prevent radiation.

### Description of the Related Art

X-ray procedures and equipment have been used for decades to create medical images by directing X-ray beams through a patient's body, whether they are a living person or an animal. Despite the enormous medical benefits, such ionizing radiation is not without risks to the patient's health. Therefore, the use of X-rays in living patients is strictly regulated by law. In particular, there are maximum permissible limits for the rate of X-ray dose reaching the patient's body surface. The closer the radiator (e.g., the focal point of the X-ray tube) is to the patient, the higher the dose rate. For this reason, a minimum distance from the radiator—the focal point to the patient's skin—was introduced. For example, it should be mentioned here that the 21st Code of Federal Regulations of the U.S. Department of Health and Human Services in DHHS 21 CFR 1020.32e defines the so-called Maximum Input Exposure Factor for the permissible skin entry dose. The use of X-ray equipment in medical diagnostics and other areas therefore requires effective control of radiation to minimize the risk of health damage to patients and medical personnel. X-ray machines with a mechanical, removable skin protection tube that increases the minimum distance to the focal point are known.

In the so-called under-table X-ray unit, the X-ray generator is placed under the patient table. The patient table is height-adjustable, i.e. the distance between the patient lying on the table or tabletop and the X-ray generator is adjustable. The physician working with the patient can adjust the table to an ergonomic working position. The legally required minimum distances to the patient's skin should be observed.

It is known that the X-ray generator can be operated permanently below its maximum power range when it is activated, i.e. with the product of tube current and acceleration voltage that is lower than the maximum that the X-ray source can actually emit.

The dose rate is automatically adjusted in the state of the art. For example, doctors can automatically access higher quality X-ray images during an operation by simply raising the treatment table to work close to the detector.

Document DE1317696U describes an X-ray sphere with a non-removable dental tube that can be used as an aiming device for aligning the central beam.

Document DE4447856C2 discloses an X-ray source whose housing is fitted with rod-shaped, non-removable spacers arranged parallel to the central beam, which serve both to protect an X-ray collimator from impacts and to ensure a minimum distance from the focal point to the skin, for example to fulfill FDA (US Food and Drug Administration) requirements.

Document DE3781171T2 describes a mobile C-arm for military applications (Philips BV 25 T), which can be dismantled into several assemblies without tools for easier transportation. From the Operation Manual for the device, English from 2020 (http://hdl.handle.net/20.500.12091/1430, source: https://www.usa.philips.com/healthcare, retrieved on 27 Apr. 2023) and from the operating instructions Philips X-ray device, field, light, BV 25 T, German with year of publication 1989 of the scanned printed copy, (retrieved on 27 Apr. 2023) it is known from FIGS. 22 and 23 and the associated descriptive text that various tubes (for fluoroscopy and dental) can be removed and replaced together with a retaining plate. It is disclosed that the X-ray device cannot be operated without the tube in place.

Document DE29520926U1 describes a tube that is integrated into a tube housing half and cannot be removed from the radiation field, in which a radiation limiting part is held in such a way that it is detachable.

From document DE60128722T2, a mobile C-arm with a tube to ensure a safe distance is provided; no option of removing the tube is disclosed.

### SUMMARY

Without limiting the scope of the present disclosure, certain advantageous features are recited in the claims of the present application.

In a first aspect, a medical imaging system includes a C-arm X-ray machine for generating X-ray radiation, the C-arm X-ray machine capable of being moved into various positions to enable different views of a patient during an imaging procedure; a table or patient support surface configured to position the patient during the imaging procedure; at least one distance sensor configured to measure a distance between the patient or the table and the C-arm X-ray device or an X-ray generator or C-arm housing of the C-arm X-ray device; and one or more processors configured by computer-executable instructions stored in a non-transitory computer-readable memory to control a radiation dose of the X-ray machine based on distances measured by the at least one distance sensor and to interrupt or reduce the radiation dose in critical situations while maintaining sufficient image quality by adjusting the radiation intensity in real time to limit radiation exposure of the patient's skin.

In some embodiments, the medical imaging system further includes a display device configured to display visual information about distance, dose control, and skin protection during the imaging procedure.

In some embodiments, the at least one distance sensor includes a capacitive sensor positioned in proximity to the X-ray generator, a C-arm of the C-arm X-ray machine, or a detector housing of the C-arm X-ray machine for sensing changes in capacitance between the sensor and a target object, and the medical imaging system further includes signal processing circuitry configured to convert the detected capacitance changes into distance values and a display device that outputs the measured distance values in a form that can be read by medical personnel.

In some embodiments, the one or more processors are further configured to analyze the measured distance information and to detect collision hazards between the C-arm X-ray machine and the patient or the table.

In some embodiments, the one or more processors are further configured to continuously analyze the measured distances and identify patterns or trends in the data, compare the identified patterns or trends in the data with predefined collision criteria, and initiate collision avoidance measures if the collision criteria are met, based on a result of the comparison.

In some embodiments, the collision avoidance measures include one or more of a warning is issued to an operator of the medical imaging system, a deceleration or stop of movement of the medical imaging system, or an automatic evasive maneuver to prevent a collision with a detected object.

In some embodiments, the one or more processors are further configured to implement anti-collision protection in all directions and implement skin protection in the direction of the radiation.

In some embodiments, the medical imaging system further includes a display device for displaying sensory collision protection and dose adjustment.

In some embodiments, the medical imaging system further includes one or more of a color-coding unit, an optical coding unit, a haptic coding unit, or an acoustic coding unit, configured to code the information shown on the display, wherein certain codes are assigned to certain dose ranges or collision scenarios.

In some embodiments, the medical imaging system further includes one or more external or internal cameras configured to monitor a C-arm position of the C-arm X-ray machine.

### BRIEF DESCRIPTION OF THE DRAWINGS

The invention will now be described in more detail with reference to the figures, which show non-limiting embodiments of the invention, wherein:

FIG. 1 schematically shows a device according to the present disclosure in the form of a mobile C-arm 11, which is intended for the implementation of the methods according to the present disclosure;

FIG. 2 shows a schematic representation of the distance measuring system;

FIGS. 3a-3c show a system for sensory collision detection with reaction mechanisms;

FIG. 4 shows a representation of the sensory collision protection system and the dose adjustment on a display of a control unit; and

FIG. 5 describes an example workflow of the system.

### DETAILED DESCRIPTION

The present disclosure offers a solution to the challenges discussed above by using sensors to measure and monitor the minimum distances as well as to adjust and/or switch off the dose rate. It is also suitable for simultaneous collision monitoring. The X-ray devices according to the present disclosure comprise an X-ray tube for generating X-rays and a detector for recording the radiation. Sensors are also integrated into the device to monitor the radiation intensity and the distance to the X-ray source and/or to detect possible collisions. The sensors can include various types of detection that are suitable for X-ray systems.

The recorded data are analyzed by a control and/or computing unit that is able to evaluate the distance to the object in real time. Based on the results, adjustments are automatically made to the X-ray machine to limit and/or completely switch off the radiation exposure. This may include adjusting the tube voltage, tube current, pulse rate and/or other parameters to reduce or eliminate radiation. In addition, the movement of the table and/or the C-arm can be adjusted to reduce or increase the corresponding distance. Furthermore, possible collisions are detected or prevented. This enables the control and/or computing unit to adaptively adjust the X-ray device to reduce and/or prevent radiation exposure and to prevent and/or avoid collisions, which minimizes the risk of damage to the health of patients and medical staff. It also helps to improve image quality by ensuring an optimum radiation dose for capturing images.

It is known that the X-ray generator can be operated permanently below its maximum power range when it is activated, i.e. with the product of tube current and acceleration voltage that is lower than the maximum that the X-ray source can actually emit. In general, the distance from the detector to the patient may be as short as possible. If this is not possible, however, sensors help to adjust the dose until the X-ray tube is switched off if the distance from the patient to the focal point of the tube is reduced or the minimum distance is not reached. Furthermore, the present technology is intended to additionally output a warning signal in acoustic, haptic or optical form.

However, the reduction of the radiation power leads to a decrease in the number of X-ray quanta generated, while at the same time increasing quantum noise. This means that there are some compromises between X-ray image quality, radiation safety and ergonomics of the overall system. An object of the present technology is to use a suitable method to ensure that the legally prescribed safety distances are maintained and that dose control (ALARA) and ergonomics are improved. ALARA=As low as reasonably achievable.

An object can be a table with or without controls, the patient himself or parts of the patient, both or other parts located in the movement area. These can also be robot-assisted systems.

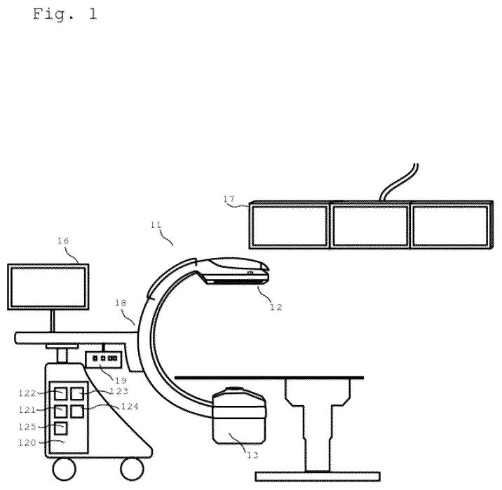

FIG. 1 schematically shows a device according to the invention in the form of a mobile C-arm 11, which is intended for the implementation of the methods according to the present disclosure.

The C-arm 11 carries an X-ray generator 13 at one end and an X-ray image detector 12, such as a flat detector or an image amplifier, at the other end and opposite the X-ray generator 13. The C-arm 11 can be adjusted under the control of a motor in a plurality of axes in space, the axes having sensors for detecting the extent of the adjustment.

The system further includes one or more computing devices 120 comprising one or more processors and non-transitory memory storing instructions that cause the one or more computing devices to implement an image-processing unit 121, a memory unit 122, a control unit 123 and a network interface 124. By means of the network interface 124, data, such as image data sets and results of the methods according to the present disclosure, can be distributed or made available in a network. The image-processing unit 121, the memory unit 122, the control unit 123, and the network interface 124 can be individual physical computing components and/or can be algorithms or functional modules executed by the one or more computing devices.

The image processing unit 121 comprises a control unit 122, on which the two- or three-dimensional image data sets used for the methods according to the present disclosure can be stored or loaded. These image data sets can either be loaded from a server or recorded by means of the C-arm 11 before or during an intervention. The memory unit further comprises instructions which are used for carrying out the methods according to the present disclosure by means of a computing unit.

Furthermore, the device can contain a graphical user interface (GUI) 19, having an image output unit (16, 17) and an input unit 19, with which corresponding settings can be made for the image processing unit 121 in corresponding organ programs.

Figure illustrates a distance measuring system with a capacitive principle of operation.

The example distance measurement system is based on the capacitive principle and is used to measure the distance between two objects. This is a non-contact-based method based on the detection of electrical capacitance changes.

The distance measurement system with capacitive principle may be used in various areas such as robotics, automation technology, vehicle technology and measurement technology. It enables precise distance measurements and helps to improve safety, accuracy and efficiency in various industrial and medical applications.

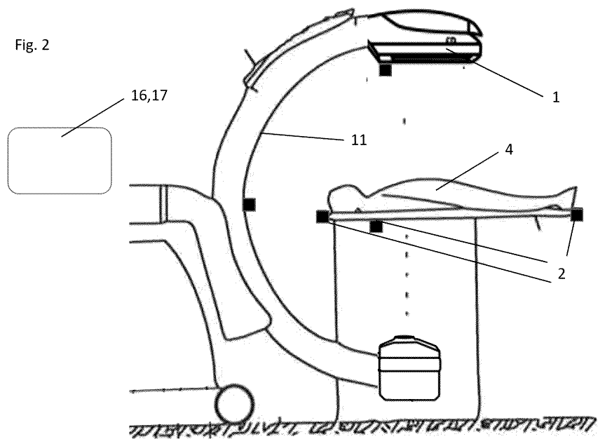

FIG. 2 shows a schematic representation of the distance measuring system.

A capacitive sensor is based on a change in the electrical capacitance of a capacitor or capacitor system. When a conductive object approaches, the electrical field of the sensor is changed, which in turn leads to a change in capacitance that can be measured. The sensor is positioned close to the objects to be measured, preferably on the generator or detector housing. The change in capacitance caused by the approach is forwarded to an evaluation unit 123, which analyses the measured values and calculates the distance between the object and the sensor. For example, it can be a single circulating sensor or several different sensors.

The capacitive distance measuring system is particularly suitable for water and metals, and therefore also for use on humans, and offers several advantages over other alternatives. Thanks to the capacitive principle, the distance measuring system can offer high accuracy and repeatability as it is independent of environmental influences such as light, temperature or dirt. The cumbersome handling of a mechanical distance tube is also eliminated. In addition, a mechanical distance tube reduces the jaw width of the C-arm, which in turn can lead to unnecessary mechanical collisions when positioning the C-arm, even when not emitting. There is also no need for storage for the distance tube.

In contrast to ultrasound, a capacitive sensor is not dependent on sound reflections and can therefore also be used in environments with strong acoustic interference. Compared to optical systems, it is less sensitive to, for example, difficult lighting conditions. Compared to inductive systems, it is less susceptible to magnetic interference. Compared to radar, it offers a higher spatial resolution and is therefore suitable for precise measurements in narrow areas.

However, all of the alternatives listed are also conceivable as sensors.

In some embodiments, there can only be one sensor 1, with all other sensors 2 shown being optional.

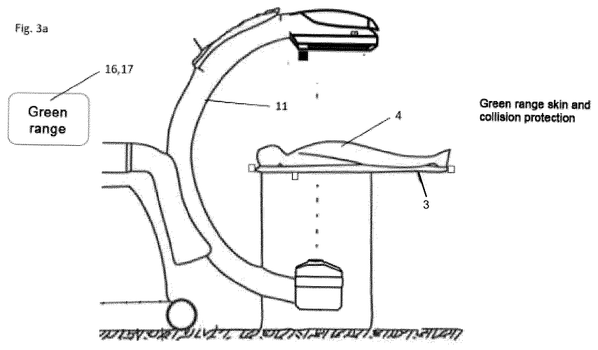

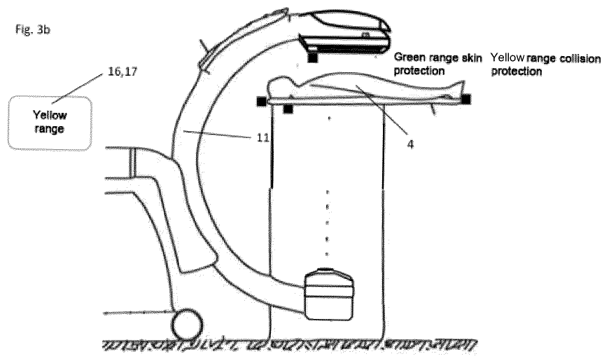

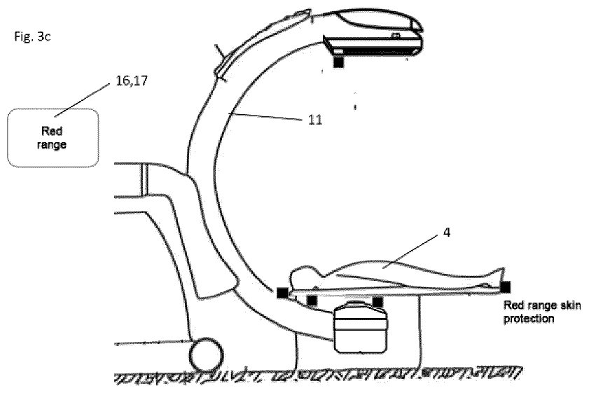

FIGS. 3a-3c show a system for sensory collision detection and the possible reactions to a detected collision. The system comprises one or more sensors, a control unit 123 and actuators for controlling the movement of an object, such as here a medical device, here in particular an X-ray device and further preferably a C-arm 11.

The sensors are strategically positioned around the object and detect its surroundings. The illustration shows various sensor types, such as ultrasonic sensors, infrared sensors, tactile sensors or cameras. Each sensor records information about the distance, obstacles or potential collisions in its detection range.

The sensor(s) can optionally be fitted in other useful positions, such as in or on the flat panel housing, in or on the C-arm directly or in or on the generator housing.

The data recorded by the sensors is forwarded to the control unit 123, which analyses the information and makes a decision on the necessary reactions.

The figures show the control unit 123 (FIG. 1) as the central unit that processes the sensor information and controls the movement of the object.

If a collision or a nearby obstacle is detected, the control unit can trigger various reactions. The illustration shows two possible reaction mechanisms: slowing down the movement and stopping the movement completely.

In the event of deceleration, the speed of the axis movement of the X-ray device and/or the object to be examined is reduced to prevent a collision by stopping the axis movement and/or adjusting the axis movement to avoid the obstacle. This can be achieved, for example, by gradually reducing the drive force and/or by changing the control parameters. This prevents a collision in good time.

In the event of a complete stop, the movement of the object is interrupted immediately. The actuators of the axis movement and/or the object, such as brakes or drive systems, are activated to ensure that the movement is stopped immediately.

The exact reaction depends on the specific requirements and the area of application. In some cases, it may also be possible to take alternative measures such as changing the direction of travel and/or maneuvering around the obstacle.

It also ensures that the legally required minimum distance from the radiator to the skin entry point is not exceeded when irradiating.

The sensor-based collision protection system with its reaction mechanisms provides effective protection against collisions and enables the X-ray machine to interact safely with its environment. It is used in a wide range of applications in the state of the art, including robotics, vehicle safety, automated production lines and other applications where collision avoidance and safety are of great importance.

FIGS. 3a-3c also show a representation of the system that ensures distance measurement, collision protection, dose control and skin protection on an X-ray machine 11, taking into account the position of the C-arm 11 and/or the table 3. The system uses various sensors and technologies to ensure the precise positioning and alignment of the C-arm and/or table during the examination.

Distance measurement: The system uses various methods for distance measurement, including monitoring the C-arm positions via appropriate encoders, capacitive sensors, external or internal cameras (such as navigation camera or similar) and pattern/image recognition. These technologies enable the system to determine the exact distance between the X-ray device 11 and the patient, the C-arm position and/or the table 3.

Collision protection: By continuously monitoring distances and positions in relation to the object, the system can recognize potential collisions. This is made possible by the integration of capacitive sensors and/or position and/or acceleration sensors. When a possible collision is detected, appropriate measures are taken to slow down and/or stop the movement and/or to avoid the obstacle/object, thus preventing injuries or damage.

Dose control: The system adjusts the radiation dose based on the information collected. In doing so, it takes into account both the position of the C-arm 11 and the position of the object 4 and/or the table 3. Using a GUI or other input unit 19 allows additional information such as the nature (material and/or thickness) of the object 4 or table 3 to be entered in order to further optimize the dose control. The dose can be evaluated and adjusted differently depending on the selected organ program or operating mode.

The organ program and the operating mode are used to define the following ranges:

Standard dose range: This range represents the standard dose used for most X-ray examinations. In this case, the radiation is emitted at a predefined dose that is considered safe and appropriate for general examinations.

Reduce dose range: In certain cases, where a lower radiation dose is sufficient or necessary for medical reasons, the radiation in this range is reduced. The system adjusts the radiation parameters to reduce the dose and expose the patient to a lower radiation dose.

Depending on the selected organ program's range: This range shows personalized dosage control tailored to the target region under investigation and different applications (e.g. organ, bone, vessel, etc.). The system takes into account the specific requirements of each target region and the desired image quality to deliver the optimal dose. In addition, the dose can be adjusted according to the distance to the object, in line with the ALARA principle.

The display 16, 17 allows the operator to monitor and control the dose in real time, thanks to the visualization of the dose status. It offers an intuitive interface to adjust the radiation dose to the specific needs of each patient and each examination, in order to optimize both image quality and safety.

Adjust radiation range: Skin protection is also integrated into the system. Based on the distance measurements and the positioning of the C-arm 11 and/or table 3, the system can evaluate skin protection and take appropriate measures to minimize radiation exposure to the skin. This may include adjusting the radiation dose or taking other appropriate measures. A warning may be issued when the minimum distance is reached and the radiation dose may be automatically reduced.

Interrupt radiation range: In situations where the minimum distance is not maintained due to the movement of the object and/or the C-arm during irradiation, radiation in this area is completely interrupted to prevent potential skin damage.

Do not expose range: This range is displayed when the minimum distance between the X-ray source and the patient's skin is undershot. The system reacts by not triggering the radiation as a way to further protect the skin. At the same time, a warning is displayed to alert the operator that the minimum distance has been critically undershot.

Warning range: This range is displayed when the distance between the X-ray source and the patient's skin, while still safe, is approaching the critical point. The system issues a warning to alert the operator to the increased danger and to urge him to take care.

Control of motor movements range: This range indicates that the system controls the motor movements of the X-ray device to avoid too narrow a gap between the X-ray source and the patient's skin. The control system ensures that the distance remains appropriate and that a dose sufficient to protect the skin is guaranteed.

The above variants are to be seen as examples and serve to provide a more detailed illustration. All other possible forms of presentation and embodiments are also conceivable.

FIGS. 3a-3c shows in a further possible embodiment the representation of a C-arm 11 with integrated position detection of the axes and various sensors that can enable precise axis positioning and alignment with the aid of suitable path detection systems. The system can use multiple technologies to accurately determine the C-arm's 11 axis position and orientation during medical examinations within the room and/or in relation to the subject.

External or internal cameras: The system can use external or internal cameras, such as a navigation camera, to determine the position of the C-arm 11. These cameras continuously capture images or videos and analyze them using pattern or image recognition algorithms to determine the exact position of the C-arm 11.

Information entered via the GUI 19: In addition to determining the position, information about the nature (material and/or thickness and/or patient information) of the object could also be entered via the system's graphical user interface (GUI) 19. This information is used to supplement the evaluation of the C-arm 11 position and to ensure the optimal configuration of the system.

Position and/or acceleration sensors: The system may be equipped with position and/or acceleration sensors to monitor the movement and alignment of the C-arm 11. These sensors continuously detect the orientation, position, acceleration and movements of the C-arm 11 and enable a precise determination of the position, speed, direction of movement and alignment.

Interface to patient table: The system can also be connected to the patient table 3 via an interface to receive information about its position and location. This enables close integration between the position of the C-arm 11 and the position of the patient on the table 3. With the coupled systems of C-arm and patient table, the activated (C-arm and operating table) axes can be moved, controlled and coordinated in order to avoid collisions and/or to bring the patient into an optimal position for image acquisition.

Furthermore, the system can be optimally and ergonomically adjusted and controlled for the intended treatments and/or workflows of the user.

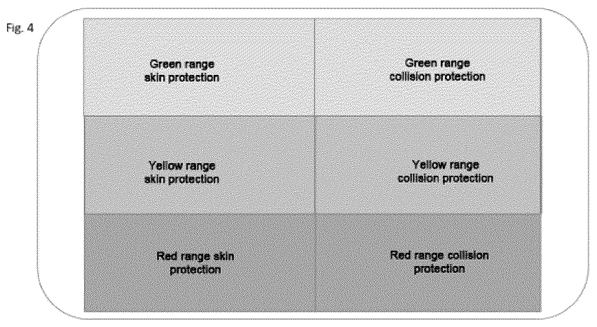

FIG. 4 shows an example representation of the sensory collision protection system and the dose adjustment on the display 16,17 of a control unit 123. The display 16,17 is used to visually indicate the distance to the object and/or potential collision points and/or dose adjustment and/or to issue warnings/messages. Information is presented as per the invention, for example using color coding, in order to clearly indicate the status of the collision protection. However, the presentation can also be acoustic, visual, haptic or other. The following diagram is an example of one possible scenario and can also be implemented in different colors and technically.

The display uses color coding to indicate the level of risk and show the appropriate reactions. Multiple ranges can be displayed, or just the critical/relevant one.

Green range: The green range represents a sufficient distance for both skin protection and collision protection. There is no immediate danger here, and the system is operating normally without making any changes to the radiation dose or the control of the axial motors.

Yellow range I: The yellow range I shows a critical but still permissible distance for skin protection. Here, the distance is so small that skin protection can no longer be seen as non-critical. The system reacts by reducing the radiation dose to ensure adequate safety according to the ALARA principle.

Yellow range II: The yellow range II indicates a critical distance for the collision. The distance to the object is too small to avoid a collision. The system reacts by slowing down the corresponding movement, thus minimizing the risk of a collision with an obstacle and/or object.

Red range I: The red range I indicates that the distance is too small to provide skin protection. Here, the critical distance is so small that even at a slowed motion, the legal minimum distance to the skin is not met. In this state, it is prohibited to generate radiation. The system reacts by interrupting the generation of radiation, and simultaneously notifies the operator and prompts him to move the device into a yellow or green range. Only then is it possible to generate radiation again.

Red range II: The red range II indicates that the distance is too small to avoid collision. There is a high probability of a collision here, even at a slower speed. The system reacts by completely stopping the axis movement and/or the movement of the object to prevent a collision. Here, too, movement is only allowed within the safe ranges.

The display allows for intuitive monitoring and control of the skin and collision protection system thanks to the visual representation of the distance and the corresponding color coding. The operator can see the status at a glance and take appropriate action to protect the patient if necessary.

Furthermore, manual intervention is possible—after confirming it is safe to do so—in order to override the system at the doctor's own discretion.

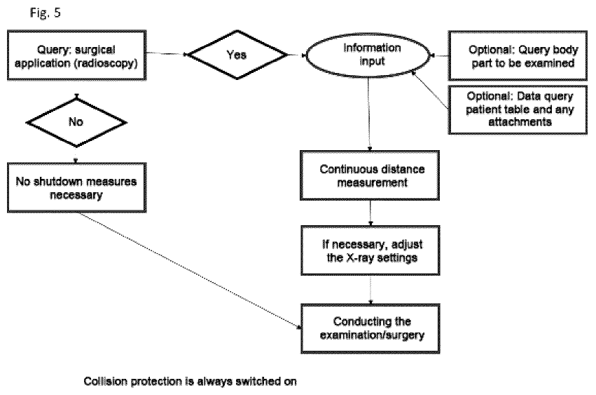

FIG. 5 describes an example workflow of the system.

The following queries are examples only and can be made via a GUI or other input methods. Repetitive processes can also be stored in the system.

Query on surgical application: The system initially checks whether the C-arm 11 is being used in a surgical application. If this is the case, the skin protection distance can be adjusted according to the normative specifications.

Optional entry of special circumstances: Optional entry of nature of the table, such as thickness, material or additional attachments to the table and/or the body region to be examined.

Distance measurement with sensors: The system uses sensors for precise distance measurement between the C-arm 11 and the part of the body being examined. The sensors detect the current distance and provide real-time data for further processing.

Taking into account patient table 3 for collision avoidance and skin protection: Depending on the positioning of the C-arm 11 (generator below or to the side of the patient table), the area to be examined is detected and, if necessary, the system takes into account the known thickness of the table 3. This ensures that the distance is used correctly for the calculations and settings.

Adjustment of X-ray settings: The system adjusts the X-ray settings based on the measured distance and the information entered regarding the nature and thickness of the body part examined and/or the patient table (object) 3. This includes, for example, the radiation intensity and exposure time to ensure an acceptable image quality while minimizing the radiation dose, or, if necessary, to abort the radiation if the minimum distance is undershot.

Performing the examination: After adjusting the X-ray settings, the examination can be performed. The C-arm 11 is moved into the desired position and the images are taken according to the defined parameters.

The workflow described is only an example and enables an efficient and safe use of a C-arm X-ray machine 11. All other conceivable and expedient workflows are equally conceivable and in accordance with the invention.

### LIST OF REFERENCE NUMBERS

1 Sensor
2 Optional table sensors
3 Table
4 Patient/Object
5 Optional sensors on the C-arm
11 C-arm
12 X-ray image detector
13 X-ray generator
16,17 Image output unit
19 Input unit
121 Image processing unit
122 Memory unit
123 Computing unit
124 Network interface

What is claimed is:

1. A medical imaging system comprising:
a C-arm X-ray machine for generating X-ray radiation, the C-arm X-ray machine capable of being moved into various positions to enable different views of a patient during an imaging procedure;
a table or patient support surface configured to position the patient during the imaging procedure;
at least one distance sensor configured to measure a distance between the patient or the table and the C-arm X-ray device or an X-ray generator or C-arm housing of the C-arm X-ray device; and
one or more processors configured by computer-executable instructions stored in a non-transitory computer-readable memory to:
control a radiation dose of the X-ray machine based on distances measured by the at least one distance sensor; and
interrupt or reduce the radiation dose in critical situations while maintaining sufficient image quality by adjusting the radiation intensity in real time to limit radiation exposure of the patient's skin.

2. The medical imaging system of claim 1, further comprising a display device configured to display visual information about distance, dose control, and skin protection during the imaging procedure.

3. The medical imaging system of claim 1, wherein the at least one distance sensor comprises a capacitive sensor positioned in proximity to the X-ray generator, a C-arm of the C-arm X-ray machine, or a detector housing of the C-arm X-ray machine for sensing changes in capacitance between the sensor and a target object, and wherein the medical imaging system further comprises:
signal processing circuitry configured to convert the detected capacitance changes into distance values; and
a display device that outputs the measured distance values in a form that can be read by medical personnel.

4. The medical imaging system of claim 1, wherein the one or more processors are further configured to analyze the measured distance information and to detect collision hazards between the C-arm X-ray machine and the patient or the table.

5. The medical imaging system of claim 1, wherein the one or more processors are further configured to:
continuously analyze the measured distances and identify patterns or trends in the data;
compare the identified patterns or trends in the data with predefined collision criteria; and
initiate collision avoidance measures if the collision criteria are met, based on a result of the comparison.

6. The medical imaging system of claim 5, wherein the collision avoidance measures comprise one or more of:
a warning is issued to an operator of the medical imaging system;
a deceleration or stop of movement of the medical imaging system; or
an automatic evasive maneuver to prevent a collision with a detected object.

7. The medical imaging system of claim 1, wherein the one or more processors are further configured to implement anti-collision protection in all directions and implement skin protection in the direction of the radiation.

8. The medical imaging system of claim 1, further comprising a display device for displaying sensory collision protection and dose adjustment.

9. The medical imaging system of claim 1, further comprising one or more of a color-coding unit, an optical coding unit, a haptic coding unit, or an acoustic coding unit, configured to code the information shown on the display, wherein certain codes are assigned to certain dose ranges or collision scenarios.

10. The medical imaging system of claim 1, further comprising one or more external or internal cameras configured to monitor a C-arm position of the C-arm X-ray machine.
